(19) United States
(12) Patent Application Publication
(10) Pub. No.: US 2026/0273113 A1
(43) Pub. Date: Sep. 17, 2026

(54) GEL COMPOSITIONS AND RELATED METHODS

(71) Applicant: Boston Scientific Scimed, Inc., Maple Grove, MN (US)

(72) Inventors: Rosangel Aimee RAMOS ESPINOZA, Boston, MA (US); Francini ZUNIGA LUNA, San Mateo, CA (US); Lauren Sfakis LYDECKER, Upton, MA (US); Carolina VILLARREAL OTALVARO, Hopedale, MA (US)

(73) Assignee: Boston Scientific Scimed, Inc., Maple Grove, MN (US)

(21) Appl. No.: 19/559,183

(22) Filed: Mar. 6, 2026

Related U.S. Application Data
(60) Provisional application No. 63/770,497, filed on Mar. 12, 2025.

(51) Int. Cl. A61K 51/12; A61K 49/22; A61K 51/04

(52) U.S. Cl. CPC A61K 51/1213; A61K 49/222; A61K 51/0406; A61K 51/1244; A61K 2123/00

(57) ABSTRACT

Compositions and related methods are described. The composition may include a polysaccharide, a crosslinker, and an imaging agent. For example, the composition may include from about 0.1 wt% to about 25 wt% of an imaging agent, based on a total weight of the composition. The polysaccharide may include chitosan or gellan gum. The imaging agent may include a radiopaque material or an echogenic material. The composition may be biocompatible and formulated as a gel.

### CROSS-REFERENCE TO RELATED APPLICATION(S)

This application claims the benefit of priority to U.S. Provisional Application No. 63/770,497, filed on March 12, 2025, which is incorporated by reference herein in its entirety.

### TECHNICAL FIELD

Aspects of this disclosure relate generally to gel compositions and related methods. In particular, aspects of this disclosure relate to gel compositions useful for imaging and/or visualization of a target site within a subject’s body.

### BACKGROUND

During medical procedures, physicians may use dyes and/or contrast agents to facilitate visualization and/or imaging of anatomy within a body of a subject. A dye may be used to mark target anatomy to assist a physician with treating the target anatomy. For example, the dye may be used to assist the physician with delivery of a drug to the target anatomy. A contrast agent may be used with an X-ray scan or a computed tomography (CT) scan to image target anatomy that cannot be seen via direct visualization. Typical dyes and contrast agents present difficulties because they can quickly disperse away from target anatomy, prompting a physician to readminister the dyes and/or contrasts agents. This may result in lengthened medical procedures and/or extended exposure to radiation.

### SUMMARY

This disclosure includes gel compositions and related methods useful for imaging and/or visualization of a target site within a subject’s body. According to some aspects, the composition comprises a polysaccharide, a crosslinker, and an imaging agent. The polysaccharide may comprise chitosan or gellan gum. The composition may comprise from about 0.1 wt% to about 25 wt% of the imaging agent, based on a total weight of the composition. The imaging agent may include a radiopaque material and/or an echogenic material. The composition may be biocompatible and formulated as a gel.

According to some aspects, the imaging agent may be distributed throughout a crosslinked matrix of the polysaccharide and the crosslinker. In some examples, the crosslinker may include a polyethylene glycol (PEG) polymer or a branched polyamine. In some examples, the PEG polymer may include a plurality of succinimidyl groups. In some examples, the branched polyamine may include trilysine. In some examples, the imaging agent may comprise a metal chosen from silver, tungsten, or tantalum. According to some aspect, the composition may comprise from about 1 wt% to about 10 wt% of the metal, based on a total weight of the composition. Additionally or alternatively, the imaging agent may comprise silver nanoparticles and glycerol, or silver nanoparticles and phosphate buffered saline. In at least one example, the composition comprises from about 3 wt% to about 10 wt% silver nanoparticles, based on a total weight of the composition. In some examples, the imaging agent comprises iodine, for example iodinated trilysine. In some aspects, the composition comprises from about 9 wt% to about 19 wt% iodinated trilysine, based on a total weight of the composition. Further, in some examples, the imaging agent comprises sulfur hexafluoride. Optionally, the composition may further comprise an accelerant. The imaging agent may be evenly distributed throughout the composition.

This disclosure also includes, for example, a composition comprising a polysaccharide, a crosslinker, and an imaging agent, wherein the polysaccharide comprises chitosan or gellan gum and/or the crosslinker comprises trilysine or a polyethylene glycol (PEG) polymer that includes a plurality of succinimidyl groups. In some aspects, the composition comprises from about 0.1 wt% to about 25 wt% of an imaging agent, based on a total weight of the composition. The imaging agent may comprise a radiopaque material that includes a metal, for example, or the imaging agent may comprise an echogenic material.

According to some aspects, the imaging agent comprises a metal that comprises silver, tungsten, or tantalum. For example, the composition may comprise from about 1 wt% to about 10 wt% of the metal, based on a total weight of the composition.

This disclosure also includes, for example, a composition comprising a polysaccharide, a crosslinker, and an imaging agent that includes silver nanoparticles. The polysaccharide may comprise chitosan or gellan gum. In some examples, the composition comprises from about 3 wt% to about 10 wt% silver nanoparticles, based on a total weight of the composition. Optionally, the imaging agent may further comprise glycerol.

### BRIEF DESCRIPTION OF THE FIGURES

The accompanying drawings, which are incorporated in and constitute a part of this application, illustrate exemplary aspects of this disclosure and together with the description, serve to explain the principles of this disclosure.

FIGS. 1A and 1B show images of porcine tissue with exemplary compositions, as discussed in Example 3.

FIGS. 2A and 2B show CT scan images of porcine tissue with exemplary compositions, as discussed in Example 3.

FIGS. 3A and 3B show ultrasound scan images of porcine tissue with exemplary compositions, as discussed in Example 3.

### DETAILED DESCRIPTION

Particular aspects of this disclosure are described in greater detail below. The terms and definitions provided herein control, if in conflict with terms and/or definitions incorporated by reference.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ± 5% of a specified amount or value. All ranges are understood to include endpoints, e.g., an amount ranging from 0.1 g to 10 g includes 0.1 g, 10 g, and all values between.

This disclosure includes biocompatible compositions formulated as gels (e.g., hydrogels) useful for imaging and/or visualization of a target site within a subject’s body, and related methods of preparing and delivering the compositions. The compositions herein may comprise a polysaccharide, crosslinker, and an imaging agent, e.g., the polysaccharide being crosslinked. The imaging agent may facilitate direct visualization of a target site and/or imaging of the target site in combination with imaging technologies, such as ultrasound scans or computed tomography (CT) scans. The compositions provided herein may be homogenous and/or have a viscosity suitable for injection to a target site within a subject’s body, among other aspects.

The compositions herein may be formulated for delivery to target sites within a subject’s body using medical devices such as syringes and/or catheters (including scope devices), among other devices useful for medical procedures. For example, the compositions herein may be injected to a target site of tissue (e.g., a tumor, a cyst, etc.), a target organ (e.g., bladder, gallbladder, liver, kidney, colon, esophagus, etc.), or various other locations within a subject’s body (e.g., biliary duct).

The compositions herein may be capable of remaining localized at a target site for an extended period of time. Localization of the composition at a desired target site of a subject for an extended period of time may reduce procedure time and/or reduce overall treatment time of the subject. For example, a physician may image and/or treat the target site of the subject during multiple visits without needing to readminister the composition.

According to some aspects of this disclosure, the composition may comprise a polysaccharide, a crosslinker, and an imaging agent. The polysaccharide and the crosslinker may form a crosslinked matrix, e.g., the imaging agent being distributed throughout the crosslinked matrix. For example, the imaging agent may be evenly distributed throughout the crosslinked matrix.

According to some aspects, the polysaccharide may comprise chitosan or gellan gum. Gellan gum is a high molecular weight anionic polysaccharide gum generally produced by microbial fermentation. Chitosan is a linear polysaccharide formed of glucosamine units derived from chitin, the structural component of crustacean exoskeletons. The compositions herein may comprise from about 0.5 wt% to about 10 wt% of the polysaccharide, such as from about 1 wt% to about 8 wt%, or from about 5 wt% to about 7.5 wt%, based on the total weight of the composition.

The structure of each polysaccharide together with the type of crosslinker influences the ability to form a crosslinked gel matrix. According to some aspects, the crosslinker may comprise a polyethylene glycol (PEG) polymer, a branched polyamine such as trilysine, or derivative thereof. For example, the PEG polymer may be functionalized with a plurality of succinimidyl groups (e.g., succinimidyl-terminated multi-arm PEG). In some examples, the branched polyamine may be in the form of a salt of an organic acid or inorganic acid. Exemplary salts suitable for the branched polyamine crosslinker may include, but not limited to, formate, acetate, propionate, butyrate, oxalate, malonate, succinate, maleate, glutarate, glycolate, lactate, malate, citrate, gluconate, halide, nitrate, phosphate, sulfate, and sulfonate. For example, the crosslinker may comprise trilysine acetate (TLA). In at least one example, the composition comprises chitosan crosslinked with a succinimidyl-terminated multi-arm PEG. In at least one example, the composition comprises gellan gum crosslinked with trilysine acetate. Optionally, the composition may comprise an accelerant to promote crosslinking. Exemplary accelerants useful for the compositions herein include, but are not limited to, sodium phosphate, sodium tetraborate, water, and combinations thereof.

As mentioned above, the compositions herein comprise an imaging agent. According to some aspects, the imaging agent may comprise a radiopaque material or an echogenic material. Optionally, the imaging agent may further comprise a dye (e.g., blue dye). The type of imaging agent may be selected based on the type of imaging contemplated. For example, radiopaque materials may be suitable for use with CT scans, echogenic materials may be suitable for use with ultrasound scans, and dyes may be suitable for direct visualization of a target site. Exemplary radiopaque materials include, but are not limited to metals, metal alloys, and compounds including iodine. For example, the imaging agent may comprise iohexol, iodinated trilysine, or polymers with iodine-containing substituents, such as PEG polymers that include iodine-containing groups. Exemplary echogenic materials include, but are not limited to, sulfur hexafluoride.

The composition may comprise from about 0.001 wt% to about 25 wt% of the imaging agent, based on a total weight of the composition, such as from about 0.1 wt% to about 25 wt%, from about 1 wt% to about 20 wt%, from about 5 wt% to about 15 wt%, from about 10 wt% to about 19 wt%, from about 1 wt% to about 8 wt%, from about 1 wt% to about 5 wt%, from about 8 wt% to about 12 wt%, from about 0.001 wt% to about 0.05 wt%, from about 0.005 wt% to about 0.02 wt% of the imaging agent. In some examples, the imaging agent may comprise iohexol or sulfur hexafluoride, and the composition may comprise from 0.001 wt% to about 0.05 wt%, such as from about 0.005 wt% to about 0.02 wt% of the imaging agent, based on a total weight of the composition.

According to some aspects, the imaging agent may comprise a metal or metal alloy. The metal or metal alloy may be in particle form. For example, the composition may comprise from about 0.5 wt% to about 20 wt% of the metal, based on a total weight of the composition, such as from about 1 wt% to about 10 wt% of the metal. The metal may be selected from silver (e.g., silver particles such as silver nanoparticles), tungsten (e.g., tungsten particles such as tungsten microparticles), or tantalum (e.g., tantalum particles such as tantalum microparticles). Optionally, the imaging agent may further comprise a medium useful to provide thorough mixing of metal particles with the other components of the composition (e.g., polysaccharide and crosslinker) during formation of the gel. Such media may include, for example, glycerol (also known as glycerin), phosphate buffered saline (PBS), or a mixture thereof.

In some examples, the composition may comprise from about 0.5 wt% to about 15 wt% of metal nanoparticles or microparticles, based on a total weight of the composition, such as from about 1 wt% to about 15 wt%, from about 3 wt% to about 10 wt%, from about 1 wt% to about 10 wt%, from about 5 wt% to about 15 wt%, or from about 8 wt% to about 10 wt%, based on a total weight of the composition. The metal nanoparticles or microparticles may comprise silver, tungsten, tantalum, or a mixture thereof.

In at least one example, the imaging agent comprises silver nanoparticles and glycerol or PBS. As mentioned above, the glycerol or PBS medium/media may facilitate distribution of the metal nanoparticles throughout the composition during formation of the gel.

The compositions herein may be formulated to crosslink before administration to a subject, or may be formulated to crosslink at a target site of a subject, e.g., during a medical procedure.

When prepared before administration to a subject, according to some aspects, the polysaccharide may be mixed with the crosslinker and the imaging agent in any suitable order. For example, the imaging agent may be added to either the polysaccharide or the crosslinker before the polysaccharide and crosslinker are combined to initiate crosslinking. In some aspects, the polysaccharide may be mixed with the crosslinker and the imaging agent to form the composition immediately before administration to a subject, e.g., via injection. In other aspects, the polysaccharide may be mixed with the crosslinker to form a mixture, followed by adding the imaging agent prior to setting of the gel. That is, the imaging agent may be added after crosslinking has begun but before a crosslinked matrix has fully formed, e.g., to ensure distribution of the imaging agent throughout the composition. Such gel compositions may be administered to a subject’s body via a syringe, for example.

When the composition is formulated for crosslinking at a target site, e.g., in vivo during a medical procedure, multiple components may be prepared and delivered to a target site at the same time or sequential, e.g., such that crosslinking occurs at the target site. For example, a first mixture may be prepared by mixing the polysaccharide with the crosslinker (e.g., for polysaccharides and crosslinkers that do not crosslink immediately), and a second mixture may be prepared by mixing the imaging agent with a suitable solution such as saline or PBS, and an accelerant to initiate crosslinking. In these aspects, the first mixture and the second mixture may crosslink when they contact each other at the target site to form the gel. The first and second mixtures may be delivered to the target site by a catheter, e.g., via separate lumens of the catheter, or may be delivered to the target site by separate medical devices, e.g., separate catheters.

### Examples

The following examples are intended to illustrate aspects of this disclosure without, however, being limiting in nature. It is understood that this disclosure encompasses additional embodiments consistent with the foregoing description and following examples. This disclosure is not limited to the examples further described below and encompasses additional conditions without departing from the scope of this disclosure.

### Example 1

Compositions in gel form were prepared from chitosan, PEG, and a radiopaque imaging agent using three different methods. In this example, the imaging agent included tungsten microparticles or tantalum microparticles.

In the first method, 0.01 g of the imaging agent was mixed with 10 mL of chitosan using a spatula. The chitosan-imaging agent mixture was then mixed with 0.6 g of PEG powder using a spatula, at which time the chitosan crosslinked with the PEG, with metal particles of the imaging agent distributed in the crosslinked gel matrix.

In the second method, 0.01 g of the imaging agent was mixed with 0.6 g of PEG powder. The PEG-imaging agent mixture was then mixed with 10 mL of chitosan using a spatula. Approximately 0.1 mL to 0.5 mL of an accelerant (sodium phosphate, sodium tetraborate, and water) was added to initiate/promote crosslinking of the chitosan crosslinked with the PEG, with metal particles of the imaging agent distributed in the crosslinked gel matrix. The accelerant was added using syringe exchanges.

In a third method, the imaging agent was mixed with 0.1 mL of an accelerant (sodium phosphate, sodium tetraborate, and water). Separately, 0.6 g of PEG powder was mixed with 10 mL of chitosan. The imaging agent-accelerant mixture and the PEG-chitosan mixture were then combined and mixed together, at which time the chitosan crosslinked with the PEG. However, the crosslinked gel was very viscous/hardened, and it was determined that the high viscosity may complicate administration to a subject.

### Example 2

Gel compositions of crosslinked chitosan-PEG and silver nanoparticles as the imaging agent were prepared. The compositions were prepared using two different methods.

In the first method, 0.03 g of silver powder was mixed with 0.2 mL of glycerol to maintain the silver particles in suspension. The silver-glycerol mixture was then mixed with 10 mL of chitosan. Then, 0.6 g of PEG powder was mixed with the imaging agent (silver-glycerol)-chitosan mixture. Approximately 0.1 mL of an accelerant (sodium phosphate, sodium tetraborate, and water) was added as needed to initiate/promote crosslinking.

In the second method, 0.08 g of silver powder was mixed with 0.35 mL of PBS to maintain the silver particles in suspension. The imaging agent (silver-PBS mixture) was then mixed with 10 mL of chitosan. Then, 0.6 g of PEG powder was mixed with the imaging agent (silver-PBS)-chitosan mixture. Approximately 0.1 mL of an accelerant (sodium phosphate, sodium tetraborate, and water) was added as needed to initiate/promote crosslinking.

The compositions that included glycerol in the imaging agent were more viscous as compared to the compositions that included PBS in the imaging agent. Mixing of the silver nanoparticles in either glycerol or PBS prevented the silver nanoparticles from clumping when mixed with the chitosan. Use of glycerol or PBS provided homogenous compositions.

### Example 3

Gel compositions of crosslinked chitosan-PEG and an imaging agent or crosslinked gellan gum-trilysine and an imaging agent were prepared. A gel composition of crosslinked gellan gum-trilysine without an imaging agent was prepared for comparison. The gel compositions were injected into samples of porcine tissue and imaged to assess visualization of the compositions.

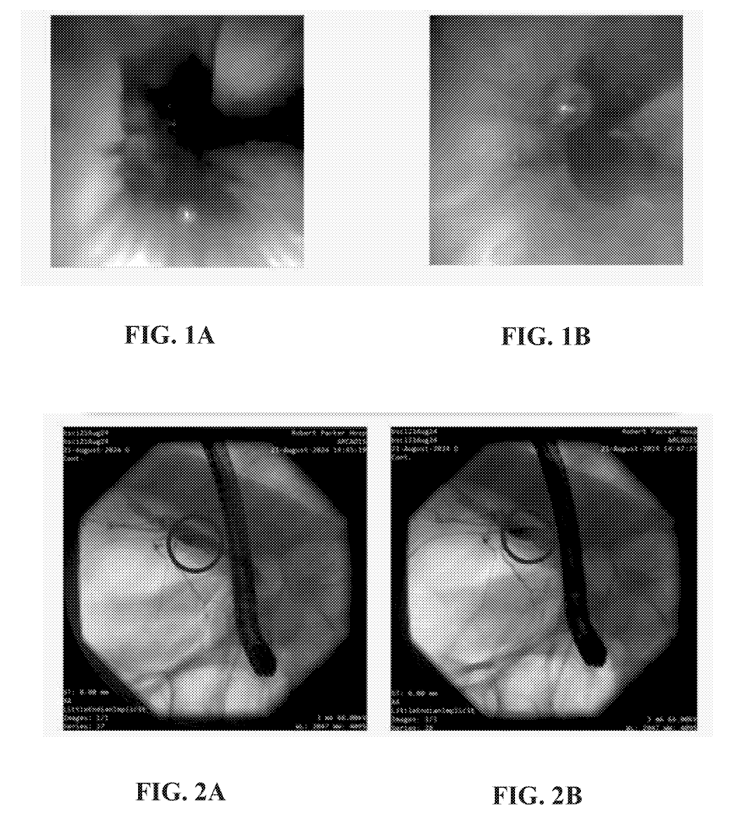

FIG. 1A is an image of porcine bladder tissue with a gel composition comprising crosslinked chitosan-PEG and blue dye as the imaging agent. FIG. 1B is an image of porcine bladder tissue with a gel composition comprising crosslinked gellan gum-trilysine without an imaging agent.

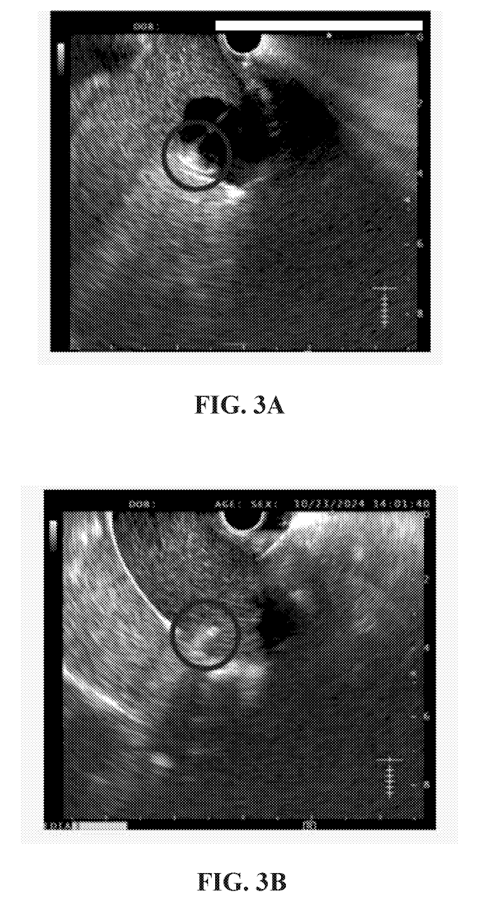

FIG. 2A is a CT scan image of porcine biliary duct tissue with a gel composition comprising crosslinked chitosan-PEG and iohexol as the imaging agent (Omnipaque™, GE HealthCare). FIG. 2B is a CT scan image of porcine biliary duct tissue with a gel composition comprising crosslinked chitosan-PEG and silver nanoparticles as the imaging agent.

FIG. 3A is an ultrasound scan image of porcine gallbladder tissue with a gel composition comprising crosslinked gellan gum-trilysine and sulfur hexafluoride as the imaging agent (SonoVue^{®,}, Bracco). FIG. 3B is an ultrasound scan image of porcine liver tissue with a gel composition comprising crosslinked gellan gum-trilysine and sulfur hexfluoride as the imaging agent (SonoVue®, Bracco).

The gel compositions with the imaging agents remained localized at the site of injection and improved visualization of the samples of porcine tissue.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A composition comprising:
a polysaccharide comprising chitosan or gellan gum;
a crosslinker; and
from about 0.1 wt% to about 25 wt% of an imaging agent, based on a total weight of the composition, wherein the imaging agent comprises a radiopaque material or an echogenic material;
wherein the composition is biocompatible and formulated as a gel.

2. The composition of claim 1, wherein the imaging agent is distributed throughout a crosslinked matrix of the polysaccharide and the crosslinker.

3. The composition of claim 1, wherein the crosslinker comprises a polyethylene glycol (PEG) polymer or a branched polyamine.

4. The composition of claim 3, wherein the PEG polymer comprises a plurality of succinimidyl groups.

5. The composition of claim 3, wherein the branched polyamine comprises trilysine.

6. The composition of claim 1, wherein the imaging agent comprises a metal chosen from silver, tungsten, or tantalum.

7. The composition of claim 6, wherein the composition comprises from about 1 wt% to about 10 wt% of the metal, based on a total weight of the composition.

8. The composition of claim 1, wherein the imaging agent comprises silver nanoparticles and glycerol, or silver nanoparticles and phosphate buffered saline.

9. The composition of claim 8, wherein the composition comprises from about 3 wt% to about 10 wt% of the silver nanoparticles, based on a total weight of the composition.

10. The composition of claim 1, wherein the imaging agent comprises iodine.

11. The composition of claim 10, wherein the imaging agent comprises iodinated trilysine.

12. The composition of claim 11, wherein the composition comprises from about 9 wt% to about 19 wt% of the iodinated trilysine, based on a total weight of the composition.

13. The composition of claim 1, wherein the imaging agent comprises sulfur hexafluoride.

14. The composition of claim 1, further comprising an accelerant.

15. The composition of claim 1, wherein the imaging agent is evenly distributed throughout the composition.

16. A composition comprising:
a polysaccharide comprising chitosan or gellan gum;
a crosslinker comprising trilysine or a polyethylene glycol (PEG) polymer that includes a plurality of succinimidyl groups; and
from about 0.1 wt% to about 25 wt% of an imaging agent, based on a total weight of the composition, wherein the imaging agent comprises a radiopaque material that includes a metal or the imaging agent comprises an echogenic material.

17. The composition of claim 16, wherein the imaging agent comprises silver, tungsten, or tantalum.

18. The composition of claim 16, wherein the composition comprises from about 1 wt% to about 10 wt% of the metal, based on a total weight of the composition.

19. A composition comprising:
a polysaccharide comprising chitosan or gellan gum;
a crosslinker; and
an imaging agent that comprises silver nanoparticles;
wherein the composition comprises from about 3 wt% to about 10 wt% of the silver nanoparticles, based on a total weight of the composition.

20. The composition of claim 19, wherein the imaging agent further comprises glycerol.
